(19) United States
(12) Patent Application Publication
(10) Pub. No.: US 2021/0198192 A1
(43) Pub. Date: Jul. 1, 2021

(54) METHODS FOR PREPARING FLORFENIOL AND INTERMEDIATE THEREOF

(71) Applicant: Heading (Nanjing) Pharmtechnologies Co., Ltd., Nanjing (CN)

(72) Inventors: Wensen LI, Nanjing (CN); Wenqi ZHANG, Nanjing (CN)

(21) Appl. No.: 16/320,213

(22) PCT Filed: Aug. 20, 2018
(86) PCT No.: PCT/CN2018/101281

§ 371 Date: Jan. 24, 2019

(30) Foreign Application Priority Data
Nov. 10, 2017 (CN) 201711101291.3

(51) Int. Cl. C07C 315/04

(52) U.S. Cl. CPC C07C 315/04; C07C 2601/16

(57) ABSTRACT

The present invention discloses a method for preparing florfenicol and its intermediate (V), comprising an addition reaction, a ring closure reaction, a hydrolysis reaction, a ring opening reaction, a reduction reaction, a ring reaction, a fluorination reaction and a ring opening reaction. In the present method for preparing florfenicol, respective reaction steps can be continuously operated, therefore the methods of the present invention features simplified process and shorter synthetic route, and obtained florfenicol has high chiral purity and is of high yield. The method of the present invention for preparing florfenicol (TM) using the intermediate (V) avoids waste water pollution and reduces the cost for treating wastewater and alleviates environmental pollution. At the same time, the methods of the present invention eliminates a chiral resolution procedure, thus increasing the utilization rate of atoms in the reaction. As a result, cost is reduced and process is simplified.

### CROSS-REFERENCE TO RELATED APPLICATIONS

The present disclosure claims priority to Chinese Patent Application No. 201711101291.3, filed on Nov. 10, 2017, the content of which is incorporated herein by reference in its entirety.

### FIELD OF THE INVENTION

The present invention belongs to the technical field of preparation of veterinary drugs, and the present invention relates to a method for preparing an intermediate (V) of florfenicol and in particular to a method for preparing florfenicol using the intermediate (V).

### BACKGROUND OF THE INVENTION

Florfenicol, also named thiamphenicol, has an alternate name of fluprofen in China. Thiamphenicol is a chloramphenicol-based broad-spectrum antibacterial drug dedicatedly used in veterinary medicine, which was successfully developed by Schering-Plough in the US in late 1980s. The florfenicol has an antibacterical activity against sensitive bacteria similar to chloramphenicol and thiamphenicol, but florfenicol is also sensitive to the bacteria that are resistant to chloramphenicol and thiamphenicol. Florfenicol was registered with the US FDA in 1996 and was approved in China. In the prevention and treatment of diseases in animals, especially in food-producing animals, florfenicol has a wide range of prospective applications.

Florfenicol has a formula of C₁₂H₁₄C₁₂FNO₄S, a molecular weight of 358.2, and a chemical structure represented by Formula (TM) as showed as follows.

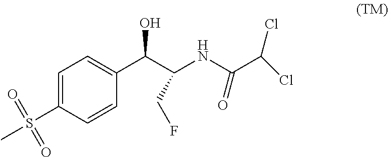 [SMILES: CS(=O)(=O)c1ccc([C@@H](O)[C@@H](CF)NC(=O)C(Cl)Cl)cc1]

At present, well-developed processes involve the use of 4-toluene sulfonyl chloride as the starting material to form p-methylsulphonyl benzaldehyde after a reduction reaction, a methylation reaction, a bromo-oxidation reaction and a hydrolysis reaction, and the p-methylsulfonyl benzaldehyde was reacted with glycine and copper(II) sulfate to form a copper salt, and the copper salt is subsequently subject to an esterification reaction and to resolution with tartaric acid to form an intermediate of D-p-methyl sulfone phenyl ethyl serinate, then D-p-methyl sulfone phenyl ethyl serinate is subject to a reduction reaction and to a reaction with dichloroacetonitrile to form an oxazoline compound, and the oxazoline compound is subject to a fluorination reaction and a hydrolysis reaction to give florfenicol. The reaction route for this process is showed as follows.

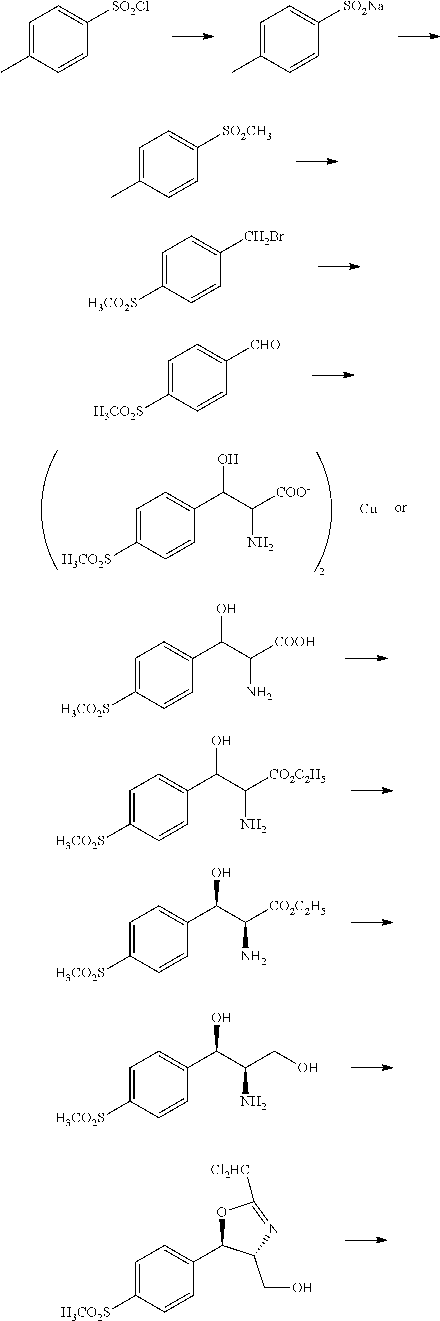

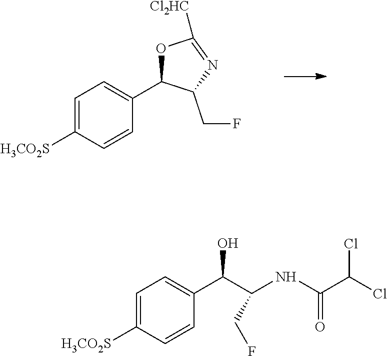

This route involves resolution of racemic D- and L-ethyl serinate, and one isomer is discarded, causing a waste of 50% of the starting materials and an increase in the manufacturing costs. Besides, a large amount of waste water of copper (II) sulfate is generated during preparation of the copper salt, which leads to a very high cost for waste water treatment and to a high environmental pressure.

In traditional synthetic processes, many by-products are formed and a low conversion rate is obtained due to the structural asymmetry of the functional groups at the chiral carbon, which leads to an increase in the cost of active pharmaceutical ingredient. Therefore, the key to reduce cost is to increase the conversion rate. The synthesis of chiral drugs requires the use of asymmetric synthesis technologies such as chiral catalysts, asymmetric catalytic synthesis, new chiral pool methods, etc., in order to improve technical aspects of the prior products and even to reduce production costs dramatically and enhance market competitiveness.

At present, with the advances in chiral synthesis technologies, asymmetric synthesis of florfenicol becomes a top priority. Studies on this type of reaction was conducted by Americans Jon E. Clark etc. using an enzyme catalysis method. Thereafter, florfenicol was synthesized by Feng Li etc. using an asymmetric synthesis method through the synthesis of an intermediate of oxazoline followed by hydrolysis and dichloroactylation. This process has the disadvantages of low yield and long synthetic route and it is difficult to be applied industrially. The reaction route is showed as follows.

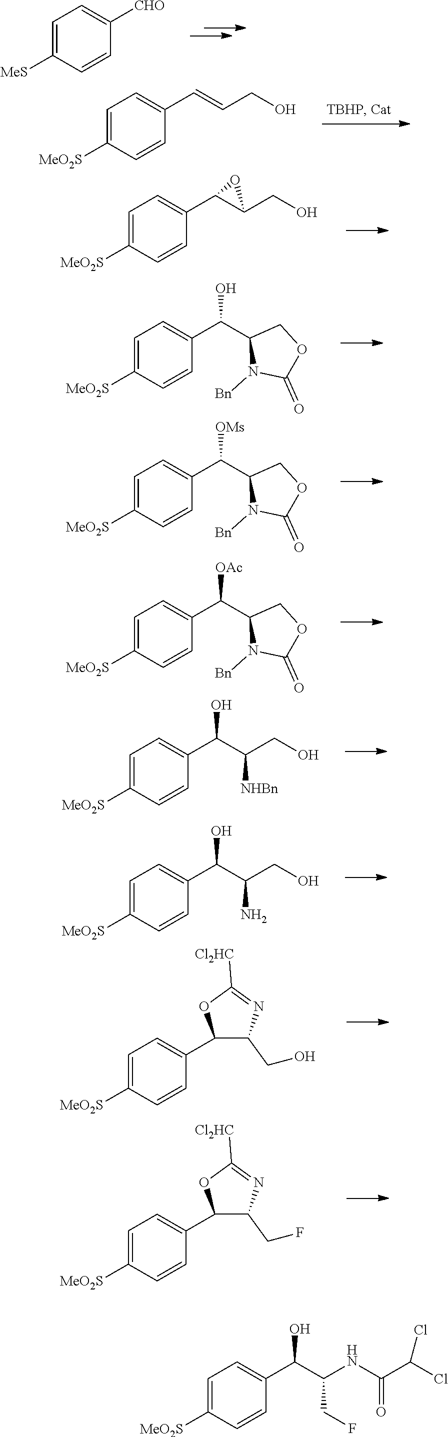

Recently, Peng Yaowu etc. conducted a study on preparation of florfenicol through asymmetric reduction using a chiral catalyst, including the use of thioanisole as the starting material, followed by sequential acylation and bromination to synthesize a substituted aziridine, and [1-substituted aziridine-2-yl][4-(methylthio)phenyl]methanone was reduced by using a synthetic chiral catalyst of trans-RuCl₂ [(R0-xylbinap] [(S)-DPEN] to form [1-substituted aziridine-2-yl][4-(methylthio)phenyl]methanol having high ee (enantiomeric excess) and de (diastereomeric excess) values; then [1-substituted aziridine-2-yl][4-(methylthio)phenyl]methanol was subject to oxidation, fluorination, deprotection and acylation to synthesize florfenicol. In this method, the use of chiral catalyst to build a chiral center carbon avoids chiral resolution. However, this process has the drawbacks that the chiral catalysts themselves are very difficult to prepare, easy to be deactivated during industrial production, and hard to recycle for repeated use, and that the starting material of thioanisole is relatively expensive, which leads to high application costs and is not conducive to industrial production. The reaction route is showed below.

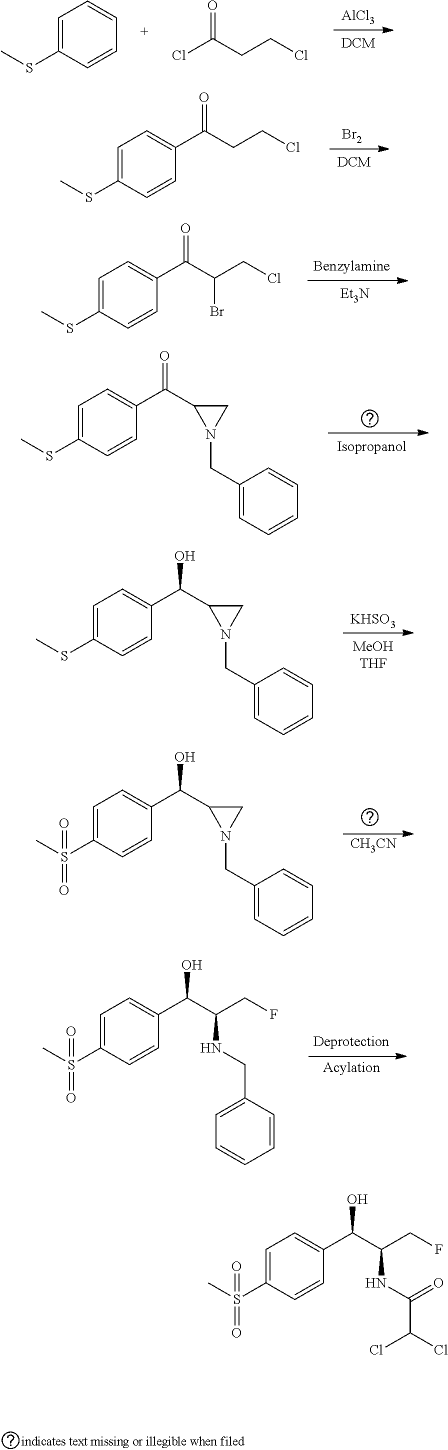

### SUMMARY OF THE INVENTION

The main object of the present invention is to provide a method for preparing an intermediate (V) of florfenicol. By using a synthetic method, chiral D-p-methyl sulfone phenyl ethyl serinate is obtained. The present invention further provides a method for preparing florfenicol using the intermediate (V). The method for preparing florfenicol features lower cost, shorter synthetic route, high yield and high chiral purity of product up to 98%.

The object of the present invention can be achieved by using the following technical solutions.

In one aspect, the present invention provides a method for preparing an intermediate (V) of florfenicol, comprising the following steps:

step (1): addition reaction

adding source material SM, compound (I) and tetraethyl titanate to a solvent of tetrahydrofuran, heating the resulting mixture and allowing the mixture to react under an atmosphere of nitrogen and under reflux to form compound (II);

step (2): ring closure reaction

adding compound (II), ethyl bromoacetate and lithium bis(trimethylsilyl)amide to a solvent of tetrahydrofuran, allowing the resulting mixture to react at a temperature in a range from −50° C. to −78° C. to form compound (III);

step (3): hydrolysis reaction

adding compound (III) to an ethanol-mixed solvent, and adding hydrochloric acid to hydrolyze compound (III) to form compound (IV);

step (4): ring opening reaction

subjecting compound (IV) to a ring opening reaction in the presence of an acid to form intermediate (V);

wherein the synthetic route for preparing the intermediate (V) of florfenicol is shown in route (1):

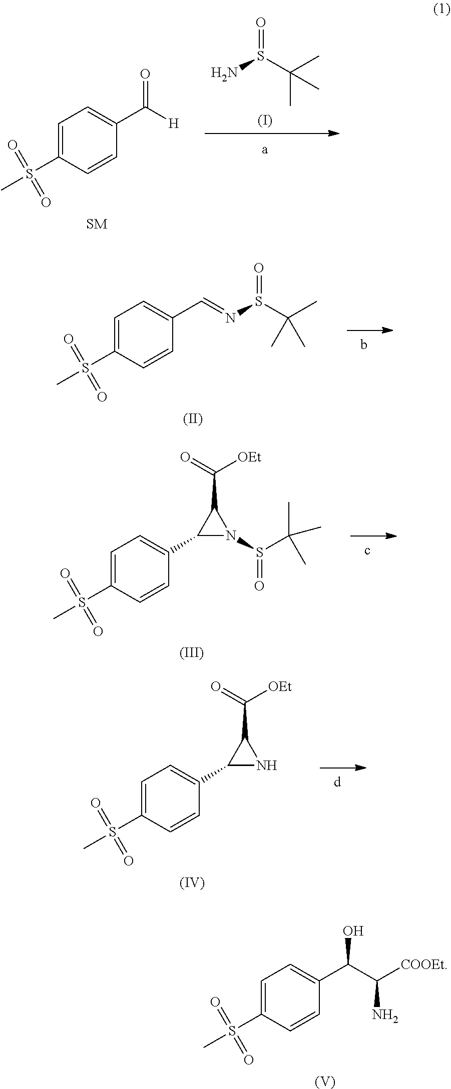

Further, in step (1), the molar ratio of the source material SM:compound (I):tetraethyl titanate is 1:1.0-1.5:1.5-2.5.

Further, in step (2), the molar ratio of compound (II):ethyl bromoacetate:lithium bis(trimethylsilyl)amide is 1:1.1-1.5:1.1-1.5.

Further, in step (3), the ethanol-mixed solvent is selected from a tetrahydrofuran-ethanol mixed solvent, an n-hexane-ethanol mixed solvent, a methyl tert-butyl ether-ethanol mixed solvent and a toluene-ethanol mixed solvent.

Further, in step (4), the acid is selected from the group consisting of glacial acetic acid, hydrochloric acid, sulfuric acid and nitric acid.

In step (1) of the method of the present invention for preparing the intermediate (V), tetraethyl titanate is reacted as a Lewis acid, one molecule of water is formed during the reaction, and tetraethyl titanate has a water absorption effect. If tetraethyl titanate is not used, slower reaction progress and incomplete reaction will be resulted. Tetraisopropyl titanate, tetrabutyl titanate, methyl titanate and the like may be an alternative option, but tetraethyl titanate is considered to be an ideal choice, because tetraethyl titanate is most commonly used and inexpensive.

In step (2) of the method of the present invention for preparing the intermediate (V), the intermediate (V) may also be in the form of methyl ester, isopropyl ester, tert-butyl ester, benzyl ester or the like. In these cases, methyl bromoacetate, isopropyl bromoacetate, tert-butyl bromoacetate or benzyl bromoacetate can be substituted for ethyl bromoacetate to form corresponding ester products. Furthermore, ethyl bromoacetate may be replaced by ethyl chloroacetate or ethyl iodoecetate, but ethyl bromide has higher activity and produces better reaction effects, it is more preferable to select ethyl bromoacetate.

In step (2) of the method of the present invention for preparing the intermediate (V), lithium bis(trimethylsilyl)amide is used for deprotonating the active hydrogen since it is a basic compound having large steric hindrance. Due to its large steric hindrance, it has a great beneficial effect on the chiral purity of the reaction product after ring closure.

In the other aspect, the present invention further provides a method for preparing florfenicol using an intermediate (V) of florfenicol, comprising the following steps:

step (1): addition reaction

adding source material SM, compound (I) and tetraethyl titanate to a solvent of tetrahydrofuran, heating the mixture and allowing the resulting mixture to react under an atmosphere of nitrogen and under reflux to form compound (II);

step (2): ring closure reaction

adding compound (II), ethyl bromoacetate and lithium bis(trimethylsilyl)amide to a solvent of tetrahydrofuran, allowing the resulting mixture to react at a temperature in a range from −50° C. to −78° C. to form compound (III);

step (3): hydrolysis reaction

adding compound (III) to an ethanol-mixed solvent, and adding hydrochloric acid to hydrolyze compound (III) to form compound (IV);

step (4): ring opening reaction

subjecting compound (IV) to a ring opening reaction in the presence of an acid to form intermediate (V);

step (5): reduction reaction

reducing intermediate (V) to compound (VI) by using a reducing agent of sodium borohydridein a solvent of ethanol;

step (6): cyclization reaction

cyclizing compound (VI) with dichloroacetonitrile in an alcohol to form an intermediate (VII);

step (7): fluorination reaction

fluorinating intermediate (VII) by using an Ishikawa reagent to form intermediate (VIII);

step (8): ring opening reaction

hydrolyzing intermediate (VIII) in an acid-containing alcohol solution to form florfenicol (TM);

wherein the synthetic route for preparing florfenicol (TM) is shown in route (2):

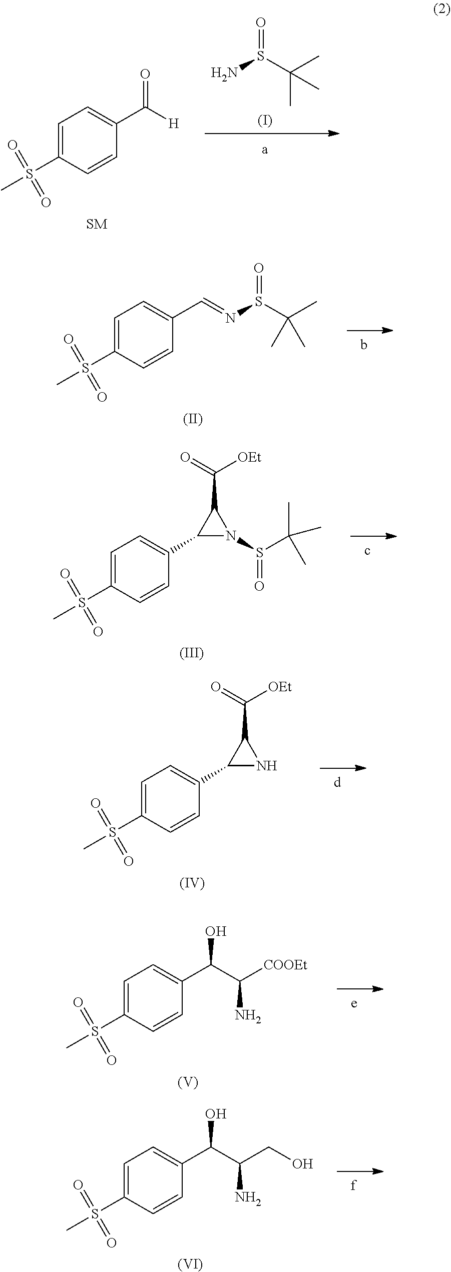

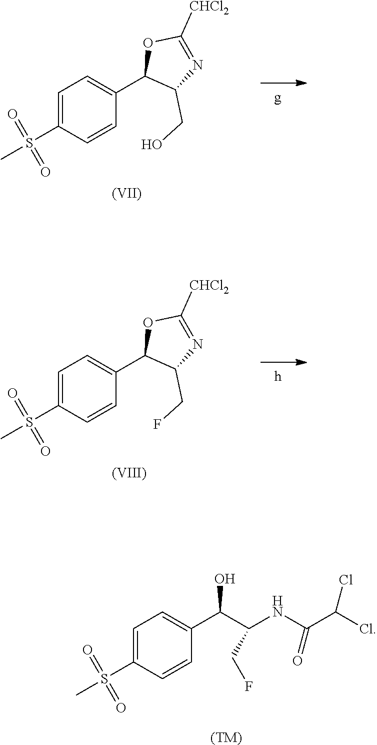

Further, in step (1), the molar ratio of the source material SM:compound (I):tetraethyl titanate is 1:1.0-1.5:1.5-2.5; and in step (2), the molar ratio of the compound (II):ethyl bromoacetate:lithium bis(trimethylsilyl)amide is 1:1.1-1.5:1.1-1.5.

Further, in step (3), the ethanol-mixed solvent is selected from a tetrahydrofuran-ethanol mixed solvent, an n-hexane-ethanol mixed solvent, a methyl tert-butyl ether-ethanol mixed solvent and a toluene-ethanol mixed solvent; and in step (4), the acid is selected from the group consisting of glacial acetic acid, hydrochloric acid, sulfuric acid and nitric acid.

Further, in step (5), the molar ratio of compound (V):sodium borohydride is 1:1.1-1.5. In step (5), since the intermediate (V) is in the form of ethyl ester, ethyl alcohol is formed during the reaction product after reduced by sodium borohydride. Thus, when ethanol is used as the solvent, it is advantageous to recover and reuse the ethanol, and at the same time, it is advantageous to reduce the formation of by-products.

In step (6), the molar ratio of compound (VI):dichloroacetonitrile is 1:1.1-1.5, and the alcohol is selected from the group consisting of methanol, ethanol, isopropanol, propylene glycol and glycerol.

Further, in step (7), the molar ratio of compound (VII):Ishikawa reagent is 1:1.1-1.5; and in step (8), the acid in the acid-containing alcohol solution is selected from the group consisting of halogenated acid and trifluoroacetic acid, and the alcohol in the acid-containing alcohol solution is selected from the group consisting of methanol, ethanol, isopropanol, propylene glycol and glycerol.

The present invention produces the following beneficial technical effects:

(1) According to the method for preparing the intermediate (V) of florfenicol and according to the method for preparing florfenicol (TM) of the present invention, the reactions from step a through step c, the reactions from step e through step f, and the reactions from step g through step h, as shown in synthetic route (2), can be continuously operated, thus the whole process has only a total of 4 steps of reaction. Therefore the process of the present invention features simplified reactions and shorter synthetic route, and florfenicol prepared as such has high chiral purity and high yield, with a total yield up to 50-60%.

(2) According to the method for preparing the intermediate (V) of florfenicol and according to the method for preparing florfenicol (TM) of the present invention, a chiral synthesis method is used to synthesize the chiral center of florfenicol, which avoids waste water pollution caused by existing processes and reduces the cost for treating wastewater and alleviates environmental pollution. At the same time, the methods of the present invention eliminates a chiral resolution procedure, thus increasing the utilization rate of atoms in the reaction. As a result, the cost is reduced and the process is simplified.

### BRIEF DESCRIPTION OF THE DRAWINGS

FIG. 1 is a ¹HNMR spectrum of the intermediate of Formula (V) prepared using the method for preparing the intermediate (V) according to the present invention;

FIG. 2 is a HPLC spectrum indicating the purity of the intermediate of Formula (V) prepared using the method for preparing the intermediate (V) according to the present invention;

FIG. 3 is a HPLC spectrum indicating the purity of the compound of Formula (VII) prepared using the method for preparing florfenicol according to the present invention;

FIG. 4 is a ¹HNMR spectrum of the compound of Formula (VII) prepared using the method for preparing florfenicol according to the present invention;

FIG. 5 a ¹HNMR spectrum of the compound of Formula (TM) prepared using the method for preparing florfenicol according to the present invention;

FIG. 6 is a ¹HNMR spectrum of the compound of Formula (II) prepared using the method for preparing the intermediate of Formula (V) according to the present invention;

FIG. 7 is a ¹HNMR spectrum of the compound of Formula (III) prepared using the method for preparing intermediate of Formula (V) according to the present invention;

FIG. 8 is a ¹HNMR spectrum of the compound of Formula (IV) prepared using the method for preparing the intermediate (V) according to the present invention;

FIG. 9 is a chiral HPLC spectrum of the intermediate of Formula (V) prepared using the method for preparing the intermediate (V) according to the present invention;

FIG. 10 is a chiral HPLC spectrum of the compound of Formula (TM) prepared using the method for preparing florfenicol according to the present invention.

### DETAILED DESCRIPTION OF THE INVENTION

In order to make the technical solutions of the present invention clearer and definite, the present invention will be further described in detail below with reference to the embodiments and the accompanying drawings, but the embodiments of the present invention are not limited thereto.

A method for preparing an intermediate (V) of florfenicol, comprising the following steps:

step (1): addition reaction

adding source material SM, compound (I) and tetraethyl titanate to a solvent of tetrahydrofuran, heating the resulting mixture and allowing the mixture to react under an atmosphere of nitrogen and under reflux to form compound (II);

step (2): ring closure reaction

adding compound (II), ethyl bromoacetate and lithium bis(trimethylsilyl)amide to a solvent of tetrahydrofuran, allowing the resulting mixture to react at a temperature in a range from −50° C. to −78° C. to form compound (III);

step (3): hydrolysis reaction

adding compound (III) to an ethanol-mixed solvent, and adding hydrochloric acid to hydrolyze compound (III) to form compound (IV);

step (4): ring opening reaction

subjecting compound (IV) to a ring opening reaction in the presence of an acid to form intermediate (V);

wherein the synthetic route for preparing the intermediate (V) of florfenicol is shown in route (1):

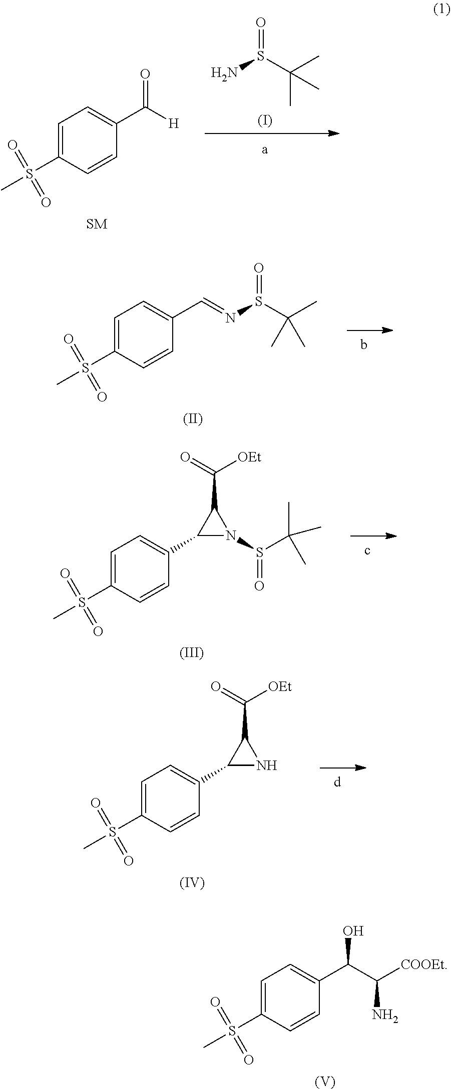

In some embodiments, in step (1), the molar ratio of the source material SM:compound (I):tetraethyl titanate is 1:1.0-1.5:1.5-2.5.

In some embodiments, in step (2), the molar ratio of compound (II):ethyl bromoacetate:lithium bis(trimethylsilyl)amide is 1:1.1-1.5:1.1-1.5.

In some embodiments, in step (3), the ethanol-mixed solvent is selected from a tetrahydrofuran-ethanol mixed solvent, an n-hexane-ethanol mixed solvent, a methyl tert-butyl ether-ethanol mixed solvent and a toluene-ethanol mixed solvent.

In some embodiments, in step (4), the acid is selected from the group consisting of glacial acetic acid, hydrochloric acid, sulfuric acid and nitric acid.

A method for preparing florfenicol using the intermediate (V), of florfenicol, comprising the following steps:

step (1): addition reaction

adding source material SM, compound (I) and tetraethyl titanate to a solvent of tetrahydrofuran, heating the mixture and allowing the resulting mixture to react under an atmosphere of nitrogen and under reflux to form compound (II);

step (2): ring closure reaction

adding compound (II), ethyl bromoacetate and lithium bis(trimethylsilyl)amide to a solvent of tetrahydrofuran, allowing the resulting mixture to react at a temperature in a range from −50° C. to −78° C. to form compound (III);

step (3): hydrolysis reaction

adding compound (III) to an ethanol-mixed solvent, and adding hydrochloric acid to hydrolyze compound (III) to form compound (IV);

step (4): ring opening reaction

subjecting compound (IV) to a ring opening reaction in the presence of an acid to form intermediate (V);

step (5): reduction reaction

reducing intermediate (V) to compound (VI) by using a reducing agent of sodium borohydride in a solvent of ethanol;

step (6): cyclization reaction

cyclizing compound (VI) with dichloroacetonitrile in an alcohol to form an intermediate (VII);

step (7): fluorination reaction

fluorinating intermediate (VII) by using an Ishikawa reagent to form intermediate (VIII);

step (8): ring opening reaction

hydrolyzing intermediate (VIII) in an acid-containing alcohol solution to form florfenicol (TM);

wherein the synthetic route for preparing florfenicol (TM) is shown in route (2):

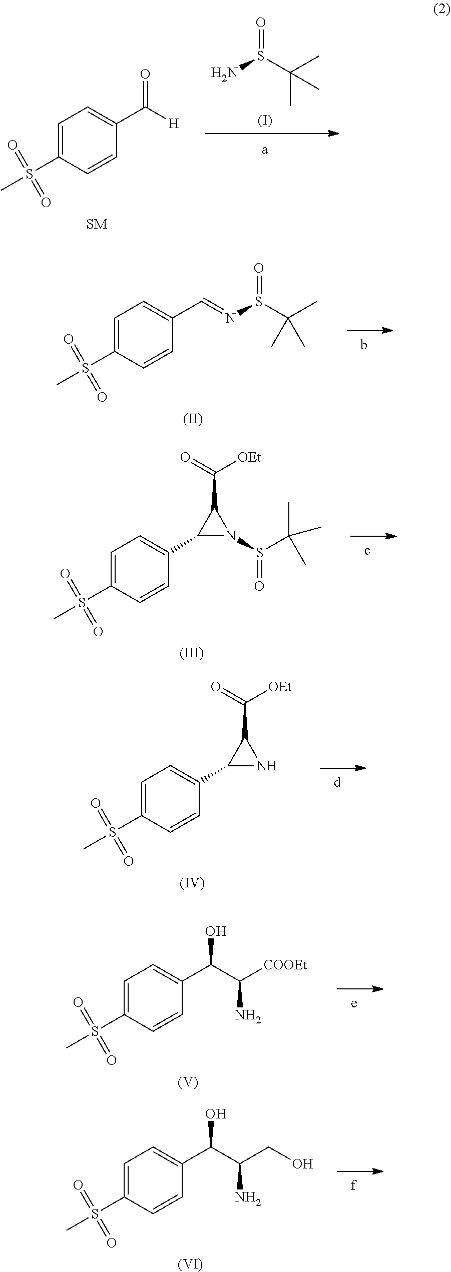

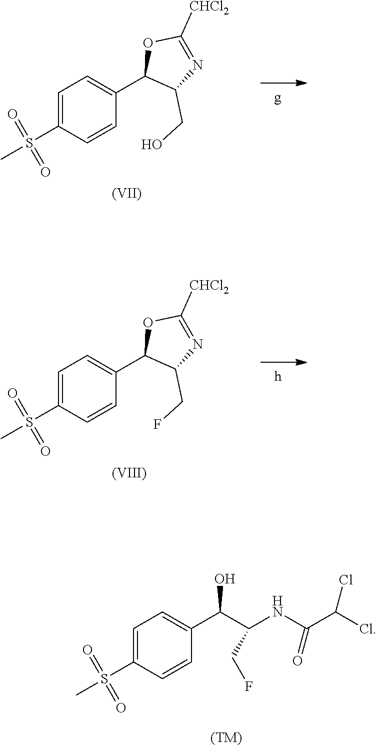

In some embodiments, in step (1), the molar ratio of the source material SM:compound (I):tetraethyl titanate is 1:1.0-1.5:1.5-2.5; and in step (2), the molar ratio of the compound (II):ethyl bromoacetate:lithium bis(trimethylsilyl)amide is 1:1.1-1.5:1.1-1.5.

In some embodiments, in step (3), the ethanol-mixed solvent is selected from a tetrahydrofuran-ethanol mixed solvent, an n-hexane-ethanol mixed solvent, a methyl tert-butyl ether-ethanol mixed solvent and a toluene-ethanol mixed solvent; and in step (4), the acid is selected from the group consisting of glacial acetic acid, hydrochloric acid, sulfuric acid and nitric acid.

In some embodiments, in step (5), molar ratio of compound (V):sodium borohydride is 1:1.1-1.5; and in step (6), the molar ratio of compound (VI):dichloroacetonitrile is 1:1.1-1.5, and the alcohol is selected from the group consisting of methanol, ethanol, isopropanol, propylene glycol and glycerol.

In some embodiments, in step (7), the molar ratio of compound (VII):Ishikawa reagent is 1:1.1-1.5; and in step (8), the acid in the acid-containing alcohol solution is selected from the group consisting of halogenated acid and trifluoroacetic acid, and the alcohol in the acid-containing alcohol solution is selected from the group consisting of methanol, ethanol, isopropanol, propylene glycol and glycerol.

### Example 1

1. Preparation of Compound (II)

In a 500 ml three-necked flask purged with nitrogen were added compound (SM) (20 g, 110 mmol), compound (I) (14.7 g, 121 mmol), tetraethyl titanate (50.2 g, 220 mmol), and THF (tetrahydrofuran) (200 ml), and the resulting mixture was heated and allowed to react under reflux for 6 hours. And the reaction was detected by liquid chromatography, after the reaction was completed, the reaction mixture was cooled to room temperature, and 60 ml of an icy solution of sodium chloride, 100 ml of ethyl acetate, 20 g of diatomaceous earth were added, then the resulting mixture was stirred for about 1 hour. Suction filtration was performed and the filtrate was extracted, and the organic phase was concentrated to afford a crude product of compound (II) (30 g) in a yield of 95%.

MS: [M+1] 288. ¹HNMR (400 MHz, CDCl₃) δ1.284 (s, 9H), 3.090 (s, 3H), 8.051-8.054 (s, 4H), 8.658 (s, 1H).

2. Preparation of Compound (III)

In a 500 ml three-necked flask were added ethyl bromoacetate (20.8 g, 125 mmol), lithium bis(trimethylsilyl)amide (20.9 g, 125 mmol), and THF (100 ml), and the resulting mixture was stirred at a temperature in the range from −50° C. to −78° C. After stirring for 30 minutes, a solution of compound (II) (30 g, 104 mmol) in tetrahydrofuran (100 ml) was added, and the temperature was controlled within the range from −50° C. to −78° C. After stirring for 1 hour, the reaction mixture was slowly warmed to room temperature, and the reaction was stirred for another 1 hour. The reaction was monitored and after completion of the reaction, water was added to quench the reaction. Ethyl acetate was added and the aqueous layer and the organic layers were separated and extraction was performed. The organic phase was washed with a solution of saturated sodium chloride, and dried over anhydrous sodium sulfate. The organic solvent was removed by rotary evaporation to give compound (III) (35 g) in a yield of 90%.

MS: [M+1] 374. ¹HNMR (300 MHz, DMSO-d₆), δ 0.906-0.953 (t, 3H), 1.495 (s, 9H), 3.219 (s, 3H).

3. Preparation of Compound (IV)

In a 500 ml three-necked flask, compound III (30 g, 80 mmol) was dissolved in a solution obtained by mixing 200 ml of tetrahydrofuran and a hydrochloric acid solution (4M) in 60 ml of ethanol, the resulting mixture was stirred at 0° C. for 5 minutes, and at 25° C.-35° C. for 1.5 hours. The reaction was monitored to the end of the reaction, and a reduced pressure distillation was carried out to give a crude product. The crude product was washed with a small amount of diethyl ether, and suction filtration was followed to give compound (IV) (18.3 g of) in a yield of 85%.

MS: [M+K] 308. ¹HNMR (300 MHz, DMSO-d₆), δ 0.967-1.013 (m, 3H), 3.26 (s, 3H), 4.004-4.095 (m, 2H), 4.986-5.014 (m, 1H), 5.207-5.236 (m, 1H), 7.902-7.919 (d, 2H), 8.022-8.049 (d, 2H), 9.021 (s, 1H).

4. Preparation of Compound (V)

In a 1000 ml three-necked flask were added compound (IV) (50 g, 186 mmol), glacial acetic acid (50 wt %, 350 ml), and dichloromethane (200 ml), the resulting mixture was stirred at 40° C.-60° C. for 3 hours and was cooled to 0° C.-10° C. The mixture was diluted with water and pH was adjusted to a value of 10. The mixture system was separated, and the aqueous phase was extracted with dichloromethane. The organic phase was collected and washed with a saturated solution of sodium chloride and dried over anhydrous sodium sulfate. The solvent was removed by evaporation under reduced pressure to give a crude product, and the crude product was washed with a small amount of diethyl ether to give compound (V) (47.5 g) in a yield of 95%.

¹H NMR (300 MHz, DMSO-d₆), δ 1.088-1.136 (t, 3H), 1.623 (s, 2H), 3.168 (s, 3H), 3.499 (d, 1H), 4.006-4.077 (m, 2H), 4.914 (t, 1H), 5.730-5.747 (d, 1H), 7.569-7.597 (d, 2H), 7.832-7.860 (d, 2H).

5. Preparation of Compound (VII)

In a 1000 ml three-necked flask were added compound (V) (50 g, 174 mmol) and ethanol (400 ml). When compound (V) was dissolved completely, sodium borohydride (7.6 g) was slowly added in batches, and the resulting mixture was heated to react under reflux for 0.5 hour. The reaction was monitored by TLC until compound (V) was completely reacted, and the reaction was terminated. Distillation was followed to distilled off 80% of the ethanol, and then glycerol (100 g) and glacial acetic acid (7.8 g) were added. After stirring for 15 minutes, dichloroacetonitrile (21.9 g) was added, and the reaction temperature was controlled at 40° C.-60° C. The mixture was reacted for 3 hours under stirring, a solid precipitated and was filtered. The solid was stirred in water to form a slurry, and the slurry was filtered to give a solid of compound (VII) (52.8 g) in a yield of 90%.

¹H NMR (400 MHz, DMSO-d₆): δ 3.228 (s, 3H), 3.55-3.61 (m, 1H), 3.68-3.78 (m, 1H), 4.05-4.11 (m, 1H), 5.17 (t, 1H), 5.75 (d, 2H), 7.257 (s, 1H), 7.589-7.609 (d, 2H), 7.979-8.000 (d, 2H).

6. Preparation of Compound (TM)

In a 1000 ml three-necked flask were added compound (VII) (50 g, 148 mmol), DCM (dichloromethane) (500 ml), and Ishikawa reagent (46.3 g, 208 mmol), and the resulting mixture was reacted for 3 hours under a pressure of 5-6 kPa. Workup was followed and organic phase was washed and concentrated to give a crude product which was directly used in the next step. In the crude product obtained in the previous step were added isopropanol (120 ml) and water (80 ml), and hydrochloric acid was added to adjust the pH value to 3-4, and the resulting mixture was reacted at a temperature of 40° C.-60° C. for 2 hours. The reaction was monitored by HPLC, after completion of the reaction, 100 ml of water was added to precipitate a solid, and suction filtration was performed to give a solid of compound (TM) (46.6 g) in a yield of 88%.

¹H NMR (400 MHz, DMSO-d₆): δ3.162 (s, 3H), 4.25-4.462 (m, 2H), 4.577-4.729 (m, 1H), 4.97 (d, 1H), 6.13 (d, 1H), 6.44 (s, 1H), 7.59 (d, 2H), 7.83 (d, 2H), 8.60 (d, 1H).

### Example 2

1. Preparation of Compound (II)

In a 1000 ml three-necked flask purged with nitrogen were added compound (SM) (40 g, 220 mmol), compound (I) (29.4 g, 242 mmol), tetraethyl titanate (100.4 g, 440 mmol), and THF (400 ml), and the resulting mixture was heated and allowed to react under reflux for 6 hours. And the reaction was detected by liquid chromatography, after the reaction was completed, the reaction mixture was cooled to room temperature, and 120 ml of an icy solution of sodium chloride, 200 ml of ethyl acetate, 50 g of diatomaceous earth were added, then the resulting mixture was stirred for about 1 hour. Suction filtration was performed and the filtrate was extracted, and the organic phase was concentrated to afford compound (II) (60 g) in a yield of 93%.

2. Preparation of Compound (III)

In a 1000 ml three-necked flask were added ethyl bromoacetate (41.6 g, 250 mmol), lithium bis(trimethylsilyl)amide (41.8 g, 250 mmol), and n-hexane (300 ml), and the resulting mixture was stirred at a temperature in the range from −50° C. to −78° C. After stirring for 30 minutes, a solution of compound (II) (60 g, 208 mmol) in n-hexane (180 ml) was added, and the temperature was controlled within the range from −50° C. to −78° C. After stirring for 1 hour, the reaction mixture was slowly warmed to room temperature, and the reaction was stirred for another 1 hour. The reaction was monitored and after completion of the reaction, water was added to quench the reaction. Ethyl acetate was added and the aqueous layer and the organic layers were separated and extraction was performed. The organic phase was washed with a solution of saturated sodium chloride, and dried over anhydrous sodium sulfate. The organic solvent was removed by rotary evaporation to give compound (III) (71.8 g) in a yield of 92%.

3. Preparation of Compound (IV)

In a 1000 ml three-necked flask, compound (III) (60 g, 160 mmol) was dissolved in a solution obtained by mixing 200 ml of tetrahydrofuran and a hydrochloric acid solution (4M) in 200 ml of ethanol, the resulting mixture was stirred at 0° C. for 5 minutes, and at 25° C.-35° C. for 1.5 hours. The reaction was monitored to the end of the reaction, and a reduced pressure distillation was carried out to give a crude product. The crude product was washed with a small amount of diethyl ether, and suction filtration was followed to give compound (IV) (43 g) in a yield of 100%.

4. Preparation of Compound (V)

In a 2000 ml three-necked flask were added compound (IV) (100 g, 372 mmol), 1 M hydrochloric acid (500 ml), and dichloromethane (400 ml), the resulting mixture was stirred at 40° C.-60° C. for 3 hours and was cooled to 0° C.-10° C. The mixture was diluted with water and pH was adjusted to a value of 10. The mixture system was separated, and the aqueous phase was extracted with dichloromethane. The organic phase was collected and washed with a saturated solution of sodium chloride and dried over anhydrous sodium sulfate. The solvent was removed by evaporation under reduced pressure to give a crude product, and the crude product was washed with a small amount of diethyl ether to give compound (V) (99.4 g) in a yield of 93%.

5. Preparation of Compound (VII)

In a 2000 ml three-necked flask were added compound (V) (100 g, 348 mmol) and ethanol (800 ml) when compound (V) was dissolved completely, sodium borohydride (15.2 g) was slowly added in batches, and the resulting mixture was heated to react under reflux for 0.5 hour. The reaction was monitored by TLC until compound (V) was completely reacted, and the reaction was terminated. Distillation was followed to distilled off 80% of the ethanol, and then isopropanol (200 g) and glacial acetic acid (15.6 g) were added. After stirring for 15 minutes, dichloroacetonitrile (43.8 g) was added, and the reaction temperature was controlled at 40° C.-60° C. The mixture was reacted for 3 hours under stirring, a solid precipitated and was filtered. The solid was stirred in water to form a slurry, and the slurry was filtered to give a solid of compound (VII) (112.9 g) in a yield of 96%.

6. Preparation of Compound (TM)

In a 2000 ml three-necked flask were added compound (VII) (100 g 296 mmol), DCM (1000 ml), and Ishikawa reagent (92.6 g, 416 mmol), and the resulting mixture was reacted for 3 hours under a pressure of 5-6 kPa. Workup was followed and organic phase was washed and concentrated to give a crude product which was directly used in the next step. In the crude product obtained in the previous step were added glycerol (250 ml) and water (160 ml), and hydroiodic acid was added to adjust the pH value to 3-4, and the resulting mixture was reacted at a temperature of 40° C.-60° C. for 2 hours. The reaction was monitored by HPLC, after completion of the reaction, 200 ml of water was added to precipitate a solid, and suction filtration was performed to give a solid of compound (TM) (99.7 g) in a yield of 94%.

### Example 3

1. Preparation of Compound (II)

In a 250 ml three-necked flask purged with nitrogen were added compound (SM) (10 g, 55 mmol), compound (I) (7.4 g, 60.5 mmol), tetraethyl titanate (25.1 g, 110 mmol), and THF (100 ml), and the resulting mixture was heated and allowed to react under reflux for 6 hours. And the reaction was detected by liquid chromatography, after the reaction was completed, the reaction mixture was cooled to room temperature, and 30 ml of an icy solution of sodium chloride, 50 ml of ethyl acetate, 10 g of diatomaceous earth were added, then the resulting mixture was stirred for about 1 hour. Suction filtration was performed and the filtrate was extracted, and the organic phase was concentrated to afford compound (II) (15.2 g) in a yield of 96%.

2. Preparation of Compound (III)

In a 250 ml three-necked flask were added ethyl bromoacetate (10.4 g, 62.5 mmol), lithium bis(trimethylsilyl)amide (10.5 g, 62.5 mmol), and methyl tert-butyl ether (100 ml), and the resulting mixture was stirred at a temperature in the range from −50° C. to −78° C. After stirring for 30 minutes, a solution of compound (II) (15 g, 52 mmol) in methyl tert-butyl ether (15 ml) was added, and the temperature was controlled within the range from −50° C. to −78° C. After stirring for 1 hour, the reaction mixture was slowly warmed to room temperature, and the reaction was stirred for another 1 hour. The reaction was monitored and after completion of the reaction, water was added to quench the reaction. Ethyl acetate was added and the aqueous layer and the organic layers were separated and extraction was performed. The organic phase was washed with a solution of saturated sodium chloride, and dried over anhydrous sodium sulfate. The organic solvent was removed by rotary evaporation to give compound (III) (17.7 g) in a yield of 91%.

3. Preparation of Compound (IV)

In a 500 ml three-necked flask, compound III (15 g, 40 mmol) was dissolved in a solution obtained by mixing 150 ml of tetrahydrofuran and a hydrochloric acid solution (4M) in 30 ml of ethanol, the resulting mixture was stirred at 0° C. for 5 minutes, and at 25° C.-35° C. for 1.5 hours. The reaction was monitored to the end of the reaction, and a reduced pressure distillation was carried out to give a crude product. The crude product was washed with a small amount of diethyl ether, and suction filtration was followed to give compound (IV) (21.5 g) in a yield of 100%.

4. Preparation of Compound (V)

In a 500 ml three-necked flask were added compound (IV) (50 g, 186 mmol), sulfuric acid (1 mol/L, 150 ml), and dichloromethane (100 ml), the resulting mixture was stirred at 40° C.-60° C. for 3 hours and was cooled to 0° C.-10° C. The mixture was diluted with water and pH was adjusted to a value of 10. The mixture system was separated, and the aqueous phase was extracted with dichloromethane. The organic phase was collected and washed with a saturated solution of sodium chloride and dried over anhydrous sodium sulfate. The solvent was removed by evaporation under reduced pressure to give a crude product, and the crude product was washed with a small amount of diethyl ether to give compound (V) (44 g) in a yield of 88%.

5. Preparation of Compound (VII)

In a 1000 ml three-necked flask were added compound (V) (50 g, 174 mmol), ethanol (400 ml), when compound (V) was dissolved completely, sodium borohydride (7.6 g) was slowly added in batches, and the resulting mixture was heated to react under reflux for 0.5 hour. The reaction was monitored by TLC until compound (V) was completely reacted, and the reaction was terminated. Distillation was followed to distilled off 80% of the ethanol, and the 1,3-propanediol (100 g) and glacial acetic acid (7.8 g) were added. After stirring for 15 minutes, dichloroacetonitrile (21.9 g) was added, and the reaction temperature was controlled within a range of 40° C.-60° C. The mixture was reacted for 3 hours under stirring, a solid precipitated and was filtered. The solid was stirred in water to form a slurry, and the slurry was filtered to give a solid of compound (VII) (53.9 g) in a yield of 92%.

6. Preparation of Compound (TM))

In a 1000 ml three-necked flask were added compound (VII) (50 g 148 mmol), DCM (500 ml), and Ishikawa reagent (46.3 g, 208 mmol), and the resulting mixture was reacted for 3 hours under a pressure of 5-6 kPa. Workup was followed and organic phase was washed and concentrated to give a crude product which was directly used in the next step. In the crude product obtained in the previous step were added 1,3-propanediol (120 ml) and water (80 ml), and hydrobromic acid was added to adjust the pH value to 3-4, and the resulting mixture was reacted at a temperature of 40° C.-60° C. for 2 hours. The reaction was monitored by HPLC, after completion of the reaction, 100 ml of water was added to precipitate a solid, and suction filtration was performed to give a solid of compound (TM) (47.7 g) in a yield of 90%.

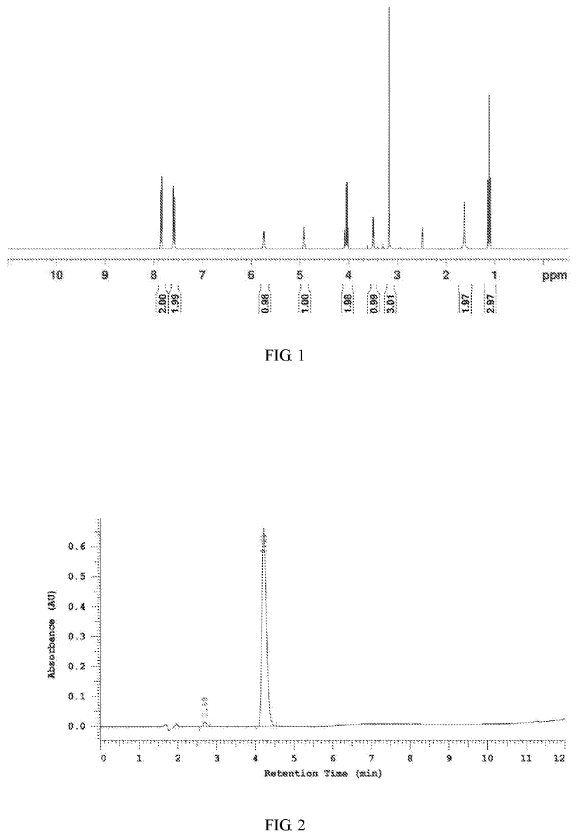

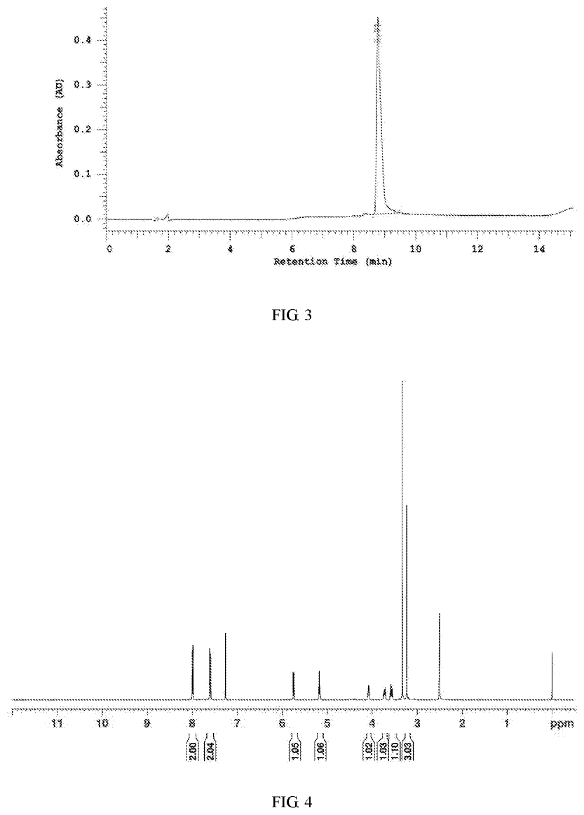

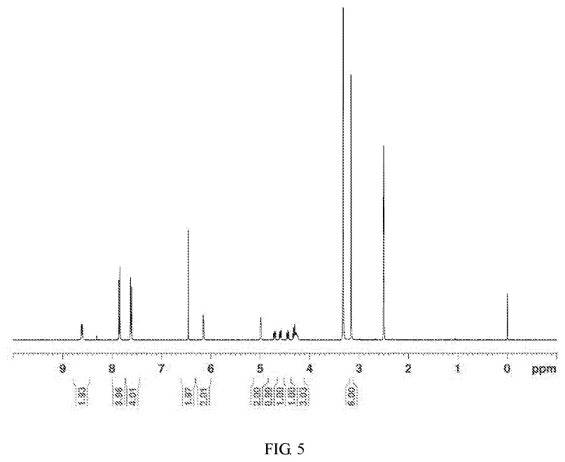

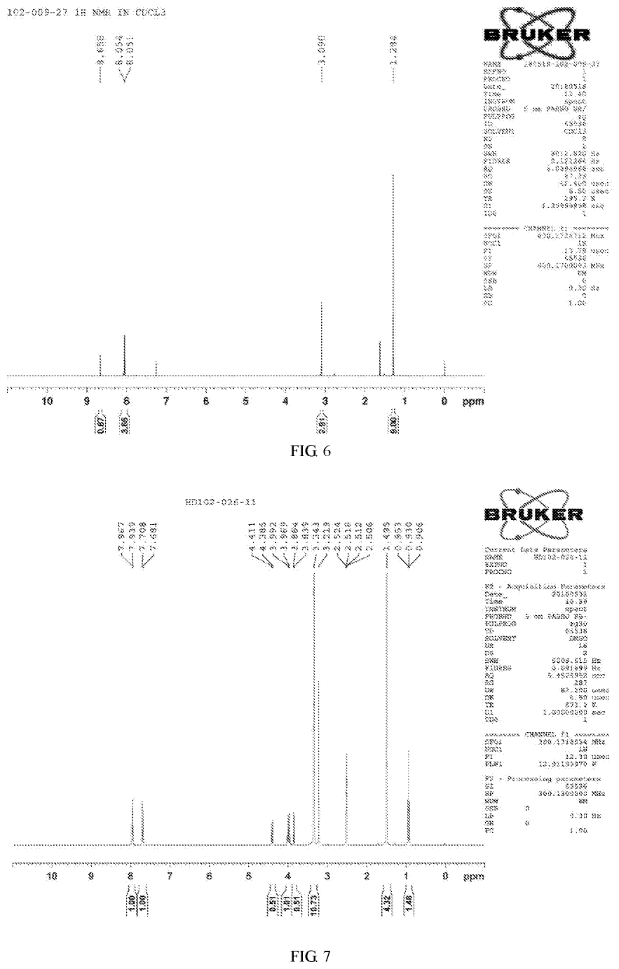

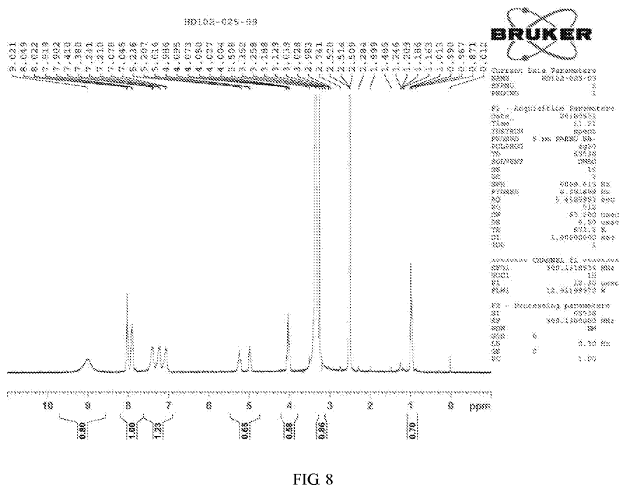

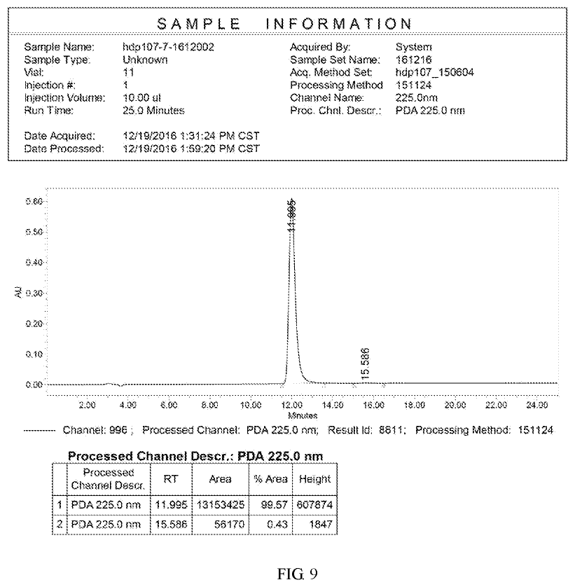

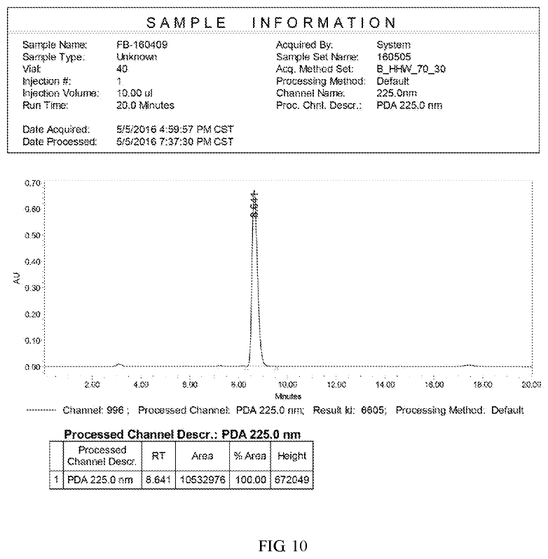

In the above examples, FIG. 1 is the ¹HNMR spectrum of the compound of Formula (V); FIG. 2 is the HPLC spectrum illustrating the purity of the compound of Formula (V); FIG. 3 is the HPLC spectrum illustrating the purity of the compound of Formula (VII); FIG. 4 is the ¹HNMR spectrum of the compound of Formula (VII); FIG. 5 is the ¹HNMR spectrum of the compound of Formula (TM); FIG. 6 is the ¹HNMR spectrum of the compound of Formula (II); FIG. 7 is the ¹HNMR spectrum of the compound of Formula (III); FIG. 8 is the ¹HNMR spectrum of the compound of Formula (IV); FIG. 9 is the chiral HPLC spectrum of the compound of Formula (V); and FIG. 10 is the chiral HPLC spectrum of the compound of Formula (TM).

As described above, according to the method for preparing the intermediate (V) of florfenicol and according to the method for preparing florfenicol (TM) in this example, the reactions from step a through step c, the reactions from step e through step f, and the reactions from step g through step h, as shown in synthetic route (2), can be continuously operated, so that the whole process has only a total of 4 steps of reaction. Therefore the process of the present invention features simplified reactions and shorter synthetic route, and florfenicol prepared as such has high chiral purity and high yield, with a total yield up to 50-60%.

According to the method for preparing the intermediate (V) of florfenicol and according to the method for preparing florfenicol (TM) in this example, a chiral synthesis method is used to synthesize a chiral center of the florfenicol, which avoids waste water pollution caused by existing processes, and reduces the cost for treating wastewater and environmental pollution. At the same time, the methods of the present invention eliminates a chiral resolution procedure, thus increasing the utilization rate of atoms in the reaction. As a result, cost is reduced and process is simplified.

The above descriptions are only preferred embodiments of the present invention, and the protection scope of the present invention is not limited thereto. Any technical person skilled in the art may make substitutions or alterations according to the disclosure of the present invention, and any equivalent substitutions or alterations within the scope of the present disclosure, and those equivalent substitutions or alterations according to the technical solution and conception of the present invention should be deemed to fall within the scope of the present invention.

What is claimed is:

1. A method for preparing an intermediate (V) of florfenicol, comprising:
step (1): addition reaction
adding source material SM, compound (I) and tetraethyl titanate to a solvent of tetrahydrofuran, heating the resulting mixture and allowing the mixture to react under an atmosphere of nitrogen and under reflux to form compound (II);
step (2): ring closure reaction
adding compound (II), ethyl bromoacetate and lithium bis(trimethylsilyl)amide to a solvent of tetrahydrofuran, allowing the resulting mixture to react at a temperature in a range from −50° C. to −78° C. to form compound (III);
step (3): hydrolysis reaction
adding compound (III) to an ethanol-mixed solvent, and adding hydrochloric acid to hydrolyze compound (III) to form compound (IV);
step (4): ring opening reaction
subjecting compound (IV) to a ring opening reaction in the presence of an acid to form intermediate (V);
wherein the synthetic route for preparing the intermediate (V) of florfenicol is shown in route (1):

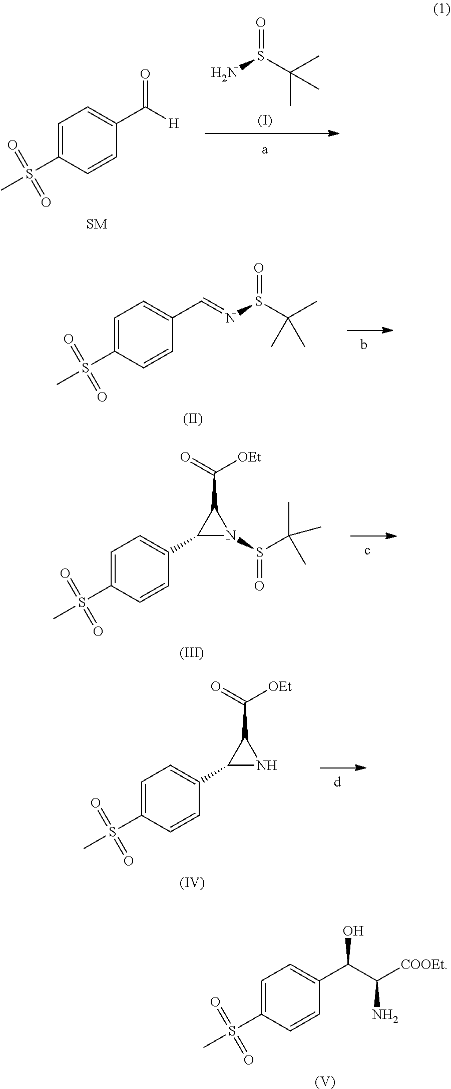

2. The method according to claim 1, wherein in step (1), the molar ratio of the source material SM:compound (I):tetraethyl titanate is 1:1.0-1.5:1.5-2.5.

3. The method according to claim 1, wherein in step (2), the molar ratio of compound (II):ethyl bromoacetate:lithium bis(trimethylsilyl)amide is 1:1.1-1.5:1.1-1.5.

4. The method according to claim 1, wherein in step (3), the ethanol-mixed solvent is selected from a tetrahydrofuran-ethanol mixed solvent, an n-hexane-ethanol mixed solvent, a methyl tert-butyl ether-ethanol mixed solvent and a toluene-ethanol mixed solvent.

5. The method according to claim 1, wherein in step (4), the acid is selected from the group consisting of glacial acetic acid, hydrochloric acid, sulfuric acid and nitric acid.

6. A method for preparing florfenicol using an intermediate (V) of florfenicol, comprising:
step (1): addition reaction
adding source material SM, compound (I) and tetraethyl titanate to a solvent of tetrahydrofuran, heating the mixture and allowing the resulting mixture to react under an atmosphere of nitrogen and under reflux to form compound (II);
step (2): ring closure reaction
adding compound (II), ethyl bromoacetate and lithium bis(trimethylsilyl)amide to a solvent of tetrahydrofuran, allowing the resulting mixture to react at a temperature in a range from −50° C. to −78° C. to form compound (III);
step (3): hydrolysis reaction
adding compound (III) to an ethanol-mixed solvent, and adding hydrochloric acid to hydrolyze compound (III) to form compound (IV);
step (4): ring opening reaction
subjecting compound (IV) to a ring opening reaction in the presence of an acid to form intermediate (V);
step (5): reduction reaction
reducing intermediate (V) to compound (VI) by using a reducing agent of sodium borohydride in a solvent of ethanol;
step (6): cyclization reaction
cyclizing compound (VI) with dichloroacetonitrile in an alcohol to form an intermediate (VII);
step (7): fluorination reaction
fluorinating intermediate (VII) by using an Ishikawa reagent to form intermediate (VIII);
step (8): ring opening reaction
hydrolyzing intermediate (VIII) in an acid-containing alcohol solution to form florfenicol (TM);
wherein the synthetic route for preparing florfenicol (TM) is shown in route (2):

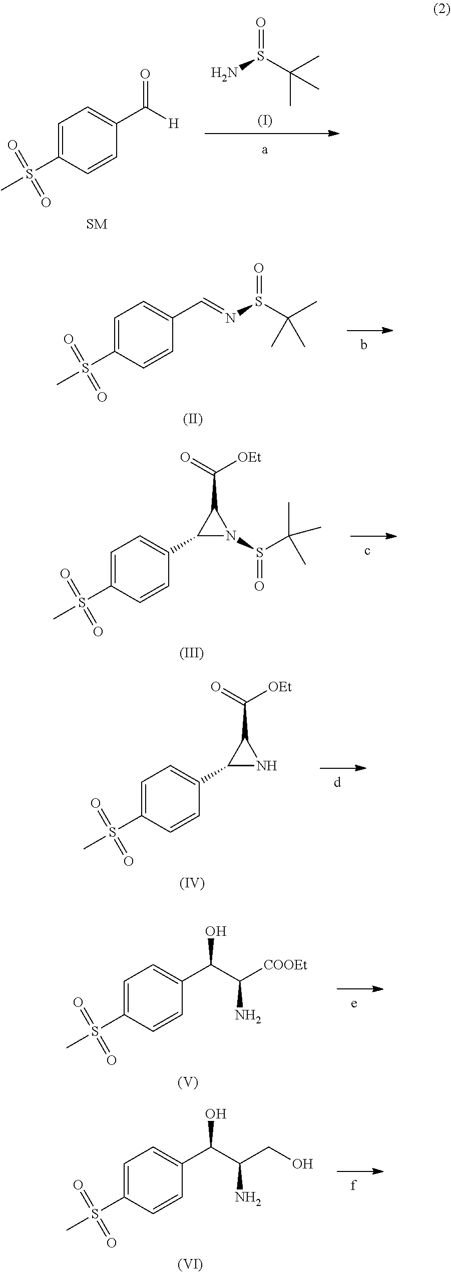

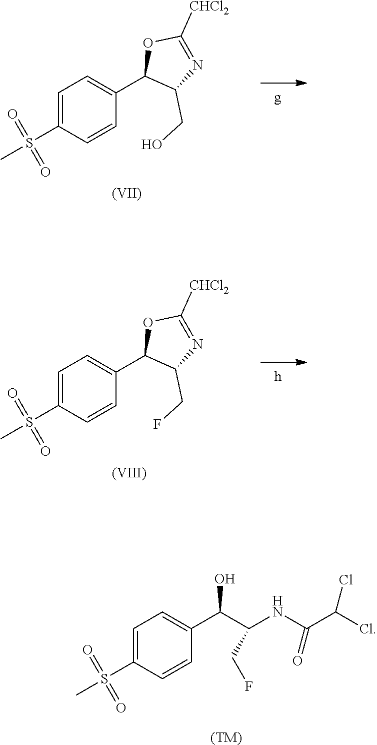

7. The method according to claim 6, wherein in step (1), the molar ratio of the source material SM:compound (I):tetraethyl titanate is 1:1.0-1.5:1.5-2.5; and in step (2), the molar ratio of compound (II):ethyl bromoacetate:lithium bis(trimethylsilyl)amide is 1:1.1-1.5:1.1-1.5.

8. The method according to claim 6, wherein in step (3), the ethanol-mixed solvent is selected from a tetrahydrofuran-ethanol mixed solvent, an n-hexane-ethanol mixed solvent, a methyl tert-butyl ether-ethanol mixed solvent and a toluene-ethanol mixed solvent;
and in step (4), the acid is selected from the group consisting of glacial acetic acid, hydrochloric acid, sulfuric acid and nitric acid.

9. The method according to claim 6, wherein in step (5), the molar ratio of compound (V):sodium borohydride is 1:1.1-1.5;
and in step (6), the molar ratio of compound (VI):dichloroacetonitrile is 1:1.1-1.5, and the alcohol is selected from the group consisting of methanol, ethanol, isopropanol, propylene glycol and glycerol.

10. The method according to claim 6, wherein in step (7), the molar ratio of compound (VII):Ishikawa reagent is 1:1.1-1.5;
and in step (8), the acid in the acid-containing alcohol solution is selected from the group consisting of halogenated acid and trifluoroacetic acid, and the alcohol in the acid-containing alcohol solution is selected from the group consisting of methanol, ethanol, isopropanol, propylene glycol and glycerol.
